(19) United States
(12) Patent Application Publication
(10) Pub. No.: US 2020/0375787 A1
(43) Pub. Date: Dec. 3, 2020

(54) URETHRAL CATHETERIZATION STRUCTURE

(71) Applicant: Wen-Hua Wang, Tainan (TW)

(72) Inventor: Wen-Hua Wang, Tainan (TW)

(21) Appl. No.: 16/872,264

(22) Filed: May 11, 2020

(30) Foreign Application Priority Data
May 29, 2019 (TW) 108206792

(51) Int. Cl. A61F 5/453; A61F 5/44; A61F 5/441

(52) U.S. Cl. CPC A61F 5/453; A61F 5/441; A61F 5/4408

(57) ABSTRACT

The present invention relates to a urethral catheterization structure for wrapping the male penis to guide urine. It comprises a urine sheath and a leaking-stop ring. A catheterization space is formed in the urine sheath, its bottom opening is connected to the catheter and urine bag. The inner side of the top opening is surrounded with a flange ring with a concave ring groove formed inside. The leaking-stop ring is a flexible foil with a wrap opening in its center, its circumference is surrounded with a collar, which can be combined with the concave ring groove tightly, so that the leaking-stop ring is expanded stably and flat, the male penis can pass through the wrap opening, and the catheterization space is isolated from the outside, preventing the urine from leaking out, and giving the expansion-shrinkage margin of erection of penis.

### FIELD OF THE INVENTION

The present invention relates generally to a urethral catheterization structure, and more particularly to a urethral catheterization structure for male penis to wear comfortably and urinate smoothly, preventing the urine from leaking, it can be removed and assembled, the comfortable sensation of wearing the urine sheath is enhanced effectively, it is leakproof and easy to clean.

### DESCRIPTION OF RELATED ART

For the long-term bedridden male patients and those who lose self-control, the urination is a big trouble. At present, the urination is handled usually by using a diaper or a urine collection device. However, the diaper wraps the lower body, the user is likely to feel wet, stuffy and hot. In order to avoid skin lesions and urinary tract infection, the diaper must be removed for observation and changed frequently, inducing troubles and inconvenience to the carers and patients. Moreover, a urine collection device is worn instead. A general urine collection device comprises a urine sheath with a catheterization space connected to a catheter, a urine bag and a urinary catheter. When in use, the opening of urine sheath covers the male penis, the glans at the front end of penis is held in the catheterization space of urine sheath, fixed by adhesive tape or bandage. During urination, the urine flows into the urine bag through the urine sheath and catheter. When the urine reaches a certain volume, the urinary catheter is opened to discharge the urine.

The urine sheath is tightened by binding adhesive tape or bandage, which is likely to have gaps as the patient's turnover causes relaxation, the urine will leak out through the gaps, causing soaking pollution, odor spilling, even drop-off of urine sheath. Therefore, in order to avoid the gaps and the drop-off of urine sheath, the adhesive tape or bandage must be bound tightly before the penis erects. However, when the penis erects, the patient feels very discomforted, even unable to urinate smoothly.

Please refer to the “urination structure” disclosed by patent bulletin No. 1520720, including a penis sheathing part, a glans protection part and a catheterization part. Wherein the penis sheathing part and catheterization part are connected to both ends of the glans protection part respectively, and the glans protection part is connected to the circumference of one end of the penis sheathing part, the top end of glans protection part converges to the circumference of end of catheterization part. There is a storage space in the glans protection part, the penis sheathing part is provided with a channel connected to the storage space, and the catheterization part is provided with a catheterization channel connected to the storage space. A spiral catheterization lug protrudes from the top end of inner wall of glans protection part to the end of inner wall of catheterization part, and the top surface of the catheterization lug is a guide bevel face tilting from the inner wall of glans protection part and catheterization part to the center. Therefore, the physically handicapped patients and those who have problems in going to toilet can urinate smoothly, so as to avoid the urine splash affecting the environment and excessive contact with skin causing skin diseases.

Said urination structure places the front end of genitals (glans) in the glans protection part to hold the penis sheathing part to support the entire urination structure. When the genitals (penis) has not erected (before erection), it is difficult to put the soft front end of genitals (glans) in the glans protection part smoothly. On the contrary, when the genitals (penis) has erected and been put in the glans protection part, the glans protection part and penis sheathing part will oppress the genitals (penis) and front end (glans) tightly, the patient will feel discomforted, even unable to urinate smoothly, meanwhile the storage space is vacuumized, so that the urine cannot flow into the urine collection bag smoothly. Furthermore, a urine collection bag is provided at the end of catheterization part, the weight of urine collection bag and the urination structure is borne only by the front end of genitals (glans). When the urine collected in the urine collection bag exceeds a certain weight or the sponge of front end of genitals (glans) contracts, the overall urination structure is disengaged, losing the practicability of wrapping. In addition, some senior citizens have atrophied or short genitals for age, disease or physical structure factors which cannot be wrapped in the urination structure.

### SUMMARY OF THE INVENTION

In order to solve said problems and defects in the urine sheath, the present invention provides a urethral catheterization structure, which can be worn by the user easily without drop-off, the comfortable sensation is enhanced and the urination is smooth, the urine will not leak, and it is easy to be removed and cleaned, guaranteeing sanitation and preventing bacterial infection.

The present invention provides a urethral catheterization structure for the male penis to wear to guide urine, comprising a urine sheath and a leaking-stop ring. A catheterization space is formed in the urine sheath, its bottom opening can be connected to the catheter and urine bag, a flange ring is provided round the inner side of top opening, a concave ring groove is formed in the flange ring. The leaking-stop ring is a flexible foil with a wrap opening in its center, surrounded by a collar, so as to form a urethral catheterization structure.

In terms of the urethral catheterization structure provided by the present invention, the collar is combined with the concave ring groove tightly, so that the leaking-stop ring is expanded stably and flat, the wrap opening provides easy wrapping and positioning of male penis, and the catheterization space is isolated from the outside, preventing the urine in the catheterization space from leaking out, and giving the penis expansion-shrinkage margin of erection, so as to avoid inappropriate binding sensation of penis, the comfortable sensation of wearing urine sheath is enhanced effectively, and it is leakproof and easy to clean.

Wherein an optionally on/off vent pipe protrudes on lateral side of the aforementioned flange ring, a connection groove to be combined with the retaining ring of the special wearing object for urine sheath is formed between the vent pipe and the flange ring.

Wherein the bottom opening of the aforementioned urine sheath converges to form a urinary catheter.

Wherein the aforementioned urine sheath is a flexible sheath.

Wherein the aforementioned urine sheath is a bending tube with a large upper opening and a small lower opening.

Wherein the urine sheath is formed integrally.

Wherein the leaking-stop ring is formed integrally.

### BRIEF DESCRIPTION OF THE DRAWINGS

FIG. 1: Stereogram of the present invention.

FIG. 2: Three-dimensional exploded diagram of the present invention.

FIG. 3: Exploded diagram of lateral section of the present invention.

FIG. 4: Combined diagram of the present invention.

FIG. 5: Schematic diagram of use of the present invention.

### DETAILED DESCRIPTION OF THE PREFERRED EMBODIMENTS

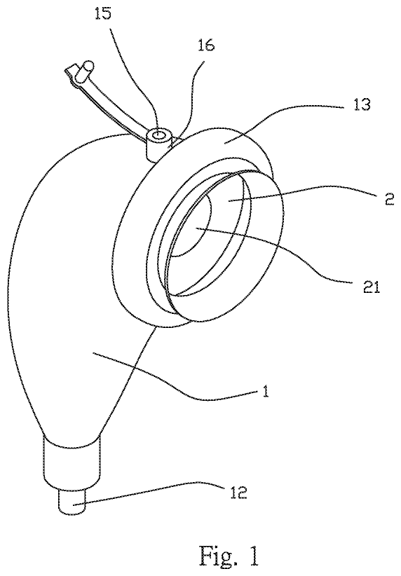

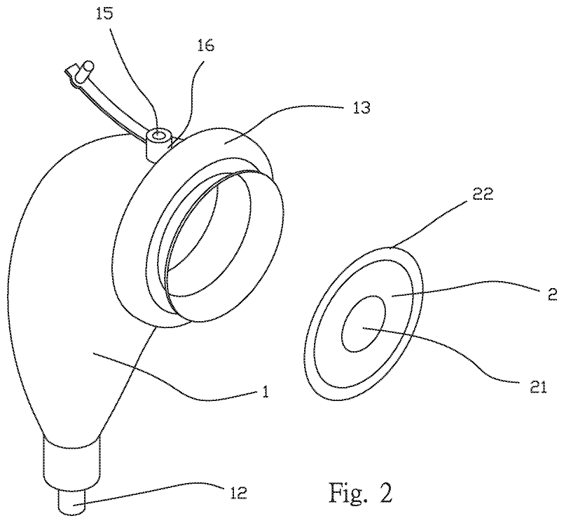

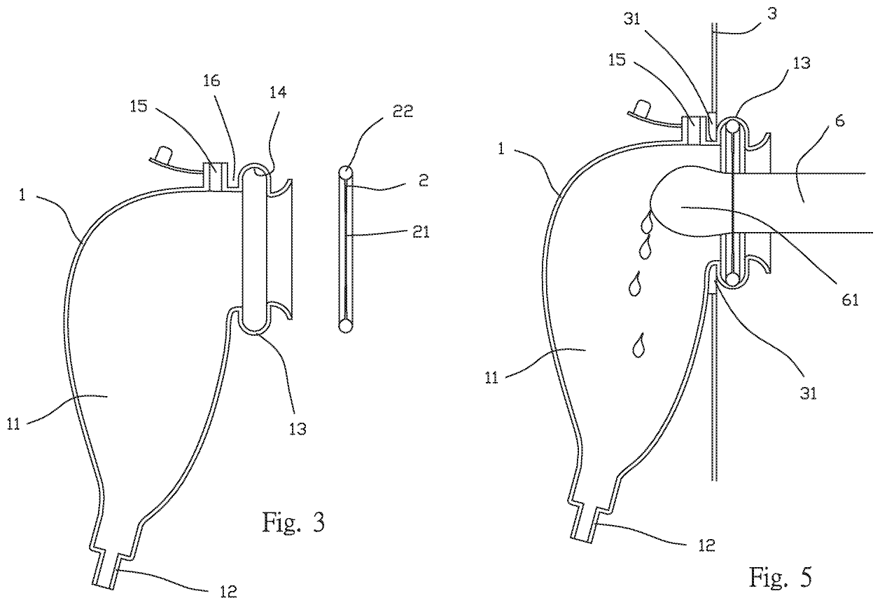

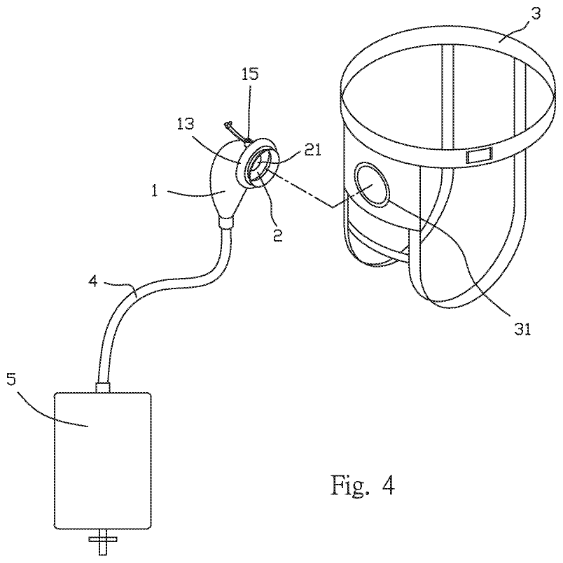

The present invention provides a urethral catheterization structure, as shown in FIGS. 1 to 4, the present invention comprises a urine sheath (1) and a leaking-stop ring (2).

The urine sheath (1) is a slightly bending tube with a large upper opening and a small lower opening integrated flexible sheath. A catheterization space (11) is formed in the urine sheath (1), the bottom opening converges to form a urinary catheter (12) connected to the catheter (4) and urine bag (5). The inner side of the top opening is surrounded with a flange ring (13). A concave ring groove (14) is formed in the flange ring (13), and an optionally on/off vent pipe (15) protrudes on the lateral side of the flange ring (13). A ring connector (16) to be combined with the retaining ring (31) of the special wearing object (3) for urine sheath is formed between the vent pipe (15) and the flange ring (13).

The leaking-stop ring (2) is an integrated flexible foil with a wrap opening (21) in its center, surrounded with a collar (22). The collar (22) can be tightly combined with the concave ring groove (14) inside the flange ring (13), so that the leaking-stop ring (2) is expanded stably and flat, and the male penis (6) can be wrapped through the wrap opening (21) (as shown in FIG. 5).

Wherein the urine sheath (1) and leaking-stop ring (2) are flexible, made of medical silica gel or other waterproof materials, so that the urine will not leak out, and the medical silica gel has soft and elastic surface, it is flexible, skin friendly, unharmful to human body and water washable. It can be washed with water after use and dried in shade, so that it can be reused until it is broken, it is environmentally friendly and economical.

Wherein the vent pipe (15) can be opened to avoid the catheterization space (11) being vacuumized, so that the urine can flow into the urine bag (5) through the catheter (4) smoothly.

To sum up, the leaking-stop ring (2) can be held in the top opening of the urine sheath (1), the collar (22) of the leaking-stop ring (2) is combined with the concave ring groove (14) of the urine sheath (1) tightly, so that the leaking-stop ring (2) can be expanded stably and flat. The ring connector (16) of urine sheath (1) is combined with a retaining ring (31) of the special wearing object (3) of urine sheath, and the urinary catheter (12) of the urine sheath (1) is connected to a catheter (4) and a urine bag (5), thus the wearing is completed, the glans (61) of male penis (6) passes through the flat expanded wrap opening (21) of leaking-stop ring (2), the wrap opening (21) wraps and positions the penis (6), whenever the user lies down, turns over or stands, the urine will not leak out during urination due to the isolation of leaking-stop ring (2), such sanitary problems as soaking pollution and odor spilling are prevented effectively, and the opening can expand or shrink moderately with the erection of penis due to the expansion-shrinkage margin of wrap opening (21), so as to avoid binding the penis (6) excessively, and the urination is more comfortable and smooth.

Further, the collar (22) of the leaking-stop ring (2) is combined with the concave ring groove (14) of the urine sheath (1), to remove and clean it, as long as it is gripped by two fingers and pulled out, the leaking-stop ring (2) can be disengaged from the urine sheath (1), the operation is convenient.

Although the invention has been explained in relation to its preferred embodiment, it is to be understood that many other possible modifications and variations can be made without departing from the spirit and scope of the invention as hereinafter claimed.

What is claimed is:

1. A urethral catheterization structure for wrapping the male penis to guide urine comprises a urine sheath and a leaking-stop ring; a catheterization space is formed in the urine sheath; the bottom of the urine sheath is opened to connect the catheter and urine bag; the inner side of the top opening of the urine sheath is surrounded with a flange ring; a concave ring groove is formed in the flange ring; the leaking-stop ring is a flexible foil with a wrap opening for wrapping the male penis in its center; the circumference of the leaking-stop ring is surrounded with a collar to be combined with the concave ring groove tightly.

2. The urethral catheterization structure according to claim 1, wherein an optionally on/off vent pipe protrudes on the lateral side of flange ring of the urine sheath, a connection groove is formed between the vent pipe and the flange ring, which is to be combined with a retaining ring of the special wearing object of urine sheath.

3. The urethral catheterization structure according to claim 1, wherein the bottom opening of the urine sheath converges to form a urinary catheter.

4. The urethral catheterization structure according to claim 1, wherein the urine sheath is a flexible sheath.

5. The urethral catheterization structure according to claim 1, wherein the urine sheath is a bending tube with a large upper opening and a small lower opening.

6. The urethral catheterization structure according to claim 1, wherein the urine sheath is formed integrally.

7. The urethral catheterization structure according to claim 1, wherein the leaking-stop ring is formed integrally.
